(19) United States
(12) Patent Application Publication
(10) Pub. No.: US 2004/0019115 A1
(43) Pub. Date: Jan. 29, 2004

(54) Use of milnacipran for the treatment of tension-type headache

(76) Inventor: Jorgen Buus Lassen, Ballerup (DK)

Correspondence Address: BIRCH STEWART KOLASCH & BIRCH, PO BOX 747, FALLS CHURCH, VA, 22040-0747, US

(21) Appl. No.: 10/446,783

(22) Filed: May 29, 2003

(30) Foreign Application Priority Data
May 30, 2002 (DK) PA 2002 00835

(51) Int. Cl. A61K 31/165

(52) U.S. Cl. 514/620

(57) ABSTRACT

This invention relates to the use of milnacipran, for the treatment of tension-type headache.

### TECHNICAL FIELD

This invention relates to the use of milnacipran, for the treatment of tension-type headache.

### BACKGROUND ART

Previously, headache disorders were not clearly distinguished and it was widely believed that they formed part of a continuum and were strongly related. In 1988, The International Headache Society, (IHS) via its ad hoc committee on classification published a document on Classification and Diagnostic Criteria for Headache Disorders, Cranial Neuralgias and Facial Pain. A new entity was here defined by name of tension-type headache.

Tension-type headache was subdivided by the IHS Classification Committee into an episodic form occurring less than half of all days and a chronic form occurring half of all days or more. Furthermore, both of these divisions were further subdivided into a form with disorder of pericranial muscle and a form without such disorder.

The classification and diagnostic criteria for tension-type headache are explained in further details in WO 98/19674 (which is hereby incorporated by reference).

Epidemiological studies have shown that chronic tension-type headache affects three per cent of the population at any given time, the lifetime prevalence being as high as six per cent. Severe episodic tension-type headache defined as persons having headache twice a week or more occurs in approximately ten per cent of the population. Thus, tension-type headache is a serious problem with significant socio-economic implications, involving enormous loss of workdays and quality of life.

Infrequent episodic tension-type headaches are usually cured by aspirin or paracetamol. However, the more frequent and severe types of episodic tension-type headache often do not respond well to plain analgesics and the patients are left virtually without effective pharmacotherapy. In chronic tension-type headache, sufferers face therapeutic problems of two kinds: Firstly, the great majority of these patients have no effect of plain analgesics and get no other therapy. Secondly, because of desperation these individuals often overconsume plain analgesics. Chronic headache is the most common reason for excessive use of plain analgesics.

Amitriptyline is the only drug with a proven prophylactic effect in chronic tension-type headache, but it helps only a minority of the patients and it only reduces headache by 30%. Furthermore, it has many side effects, such as sedation, weight gain and dryness of mouth.

WO 98/19674 describes a method for treating tension-type headache by interacting with neuronal transmission in relation to pain in connection with headache in a way that prevents or decreases central sensitization.

WO 01/62236 describes the combination of a norepinephrine reuptake inhibitor and an antimuscarinic agent.

However, there is a continued requirement to develop more selective and effective therapies that are better tolerated, for the treatment of patients with with tension-type headache.

### DETAILED DISCLOSURE OF THE INVENTION

According to the invention it has now been found that milnacipran can be used for the treatment of tension-type headache.

Thus, in its first aspect, the invention relates to the use of milnacipran or a pharmaceutically acceptable salt thereof for the manufacture of a medicament for the treatment, prevention or alleviation of tension-type headache in a subject.

In another aspect the invention relates to a method of treatment, prevention or alleviation of tension-type headache in a subject, which method comprises administering to said subject a therapeutically effective amount of milnacipran or a pharmaceutically acceptable salt thereof.

The subject to be treated according to this invention is a living body, preferably a mammal, most preferably a human, in need for such treatment.

In a further embodiment, the pharmaceutically acceptable salt of milnacipran is milnacipran hydrochloride.

In a still further embodiment of the invention, the tension-type headache to be treated, prevented or alleviated is of the type chronic tension-type headache.

Milnacipran

The hydrochloride salt of milnacipran, (Z)-1-diethylaminocarbonyl-2-aminomethyl-1-phenyl-cyclopropane, is described in U.S. Pat. No. 4,478,836 (example 4). Moret et al. in Neuropharmacology 24, 1211-19 (1985) describe its pharmacological activities and its use as an antidepressant drug.

Pharmaceutically Acceptable Salts

The active compound for use according to the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate derived, the phthalate, the salicylate, the sorbate, the stearate, the succinate, the tartrate, the toluene-p-sulphonate, and the like. Such salts may be formed by procedures well known and described in the art.

Metal salts of a chemical compound of the invention include alkali metal salts, such as the sodium salt of a chemical compound of the invention containing a carboxy group.

Pharmaceutical Compositions

While the active compound for use in therapy according to the invention may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound for use according to the invention, or a pharmaceutically acceptable salt or derivative thereof, together with one or more pharmaceutically acceptable carriers therefor, and, optionally, other therapeutic and/or prophylactic ingredients, know and used in the art. The carrier(s) must be “acceptable” in the sense of being compatible with the other ingredients of the formulation and not harmful to the recipient thereof.

The pharmaceutical composition of the invention may be administered by any convenient route which suit the desired therapy. Preferred routes of administration include oral administration, in particular in tablet, in capsule, in drage, in powder, or in liquid form, and parenteral administration, in particular cutaneous, subcutaneous, intramuscular, or intravenous injection. The pharmaceutical composition may be prepared by the skilled person using standard and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depend on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 1000 mg of active ingredient per individual dose, preferably of from about 1 to about 100 mg, are suitable for therapeutic treatments.

The active ingredient may be administered in one or several doses per day. Preferred ranges are from 10-200 mg/day p.o. administered in one or two doses, such as from 25-50 mg p.o. twice a day.

Methods of Therapy

The efficacy of use of the compound according to the invention can be evaluated by standard in vivo studies as e.g. described by Bendtsen L, Jensen R, Olesen J, in J Neurol Neurosurg Psychiatry 61, 285-290 (1996).

Combined Therapy

The compound for use according to the invention may be used or administered in combination with one or more additional drugs useful for the treatment, prevention or alleviation of tension-type headache. Such additional drug or drugs may be selected from plain analgesics such as ibuprofen, tolfenamic acid, aspirin or acetaminophen.

1. The use of milnacipran or a pharmaceutically acceptable salt thereof for the manufacture of a medicament for the treatment, prevention or alleviation of tension-type headache in a subject.

2. The use according to claim 1, wherein the tension-type headache to be treated, prevented, or alleviated is of the type chronic tension-type headache.

3. The use according to claims 1 or 2, wherein the pharmaceutically acceptable salt of milnacipran is milnacipran hydrochloride.

4. A method of treatment, prevention or alleviation of tension-type headache in a subject, which method comprises administering to said subject a therapeutically effective amount of milnacipran or a pharmaceutically acceptable salt thereof.
